(19) United States
(12) Patent Application Publication
(10) Pub. No.: US 2024/0286130 A1
(43) Pub. Date: Aug. 29, 2024

(54) STRUCTURED BIOCHIP FOR LABEL-FREE SENSING

(71) Applicants: Zethea Inc., Redwood City, CA (US); Institute of Nanoscience & Nanotechnology, Agia Paraskevi (GR)

(72) Inventors: Ioannis Raptis, Ag. Paraskevi Attikis (GR); Nikolaos Papanikolaou, Ag. Paraskevi (GR); Theofilos Kotseroglou, Hillsborough, CA (US)

(21) Appl. No.: 18/587,577

(22) Filed: Feb. 26, 2024

Related U.S. Application Data
(60) Provisional application No. 63/448,138, filed on Feb. 24, 2023.

(51) Int. Cl. B01L 3/00

(52) U.S. Cl. CPC B01L 3/502715; B01L 2300/0627; B01L 2300/0819; B01L 2300/168

(57) ABSTRACT

A structured biochip may include a structured film disposed on a substrate, where the structured film includes a plurality of sensing regions spatially distributed across the substrate, and where the plurality of sensing regions provides spatially-varying optical characteristics across the substrate. The structured film may be functionalized with binding molecules configured to selectively bind with one or more analytes, where binding of the one or more analytes to the binding molecules generates an adlayer, and where a thickness of the adlayer impacts the spatially-varying optical characteristics of the plurality of sensing regions. At least one of a presence or a concentration of at least one of the one or more analytes may be determinable based on the spatially-varying optical characteristics of the plurality of sensing regions.

### CROSS-REFERENCE TO RELATED APPLICATION

The present application claims the benefit under 35 U.S.C. § 119(e) of U.S. Provisional Application Ser. No. 63/448,138, filed Feb. 24, 2023, which is incorporated herein by reference in the entirety.

### TECHNICAL FIELD

The present disclosure is generally directed to biosensing and, more particularly, to label-free biosensing based on reflectivity variations of a structured biochip.

### BACKGROUND

In the last decades, there has been intense research activity in the development of advanced and cost-effective bioanalytical systems based on label-free optical biosensors. Due to their high detection sensitivity, short assay duration and compact size, such systems have a high potential for applications at the Point-of-Need (PoN), thus diminish the need for expensive and time-consuming laboratory analysis.

A variety of different optical sensing principles, such as planar waveguides, plasmons, broad spectrum interferometry and optical fibers have been so far employed to quantitatively determine the concentration of analytes in biological, food or environmental samples. In most of those principles, the biomolecular layer that grows on the sensor's surface during the bioreaction is detected as a shift in the recorded spectrum. Despite the advantages of these sensors, the currently available commercial systems are not suitable for use outside a laboratory. For example, Surface Plasmon Resonance (SPR), despite its widespread adoption, suffers from several limitations such as the refractive index variation of the liquid medium where the bioreactions take place, a limited number of bioreactions could be monitored simultaneously, and the high cost of the disposable sensor surface.

On the other hand, commercial devices based on reflected light spectroscopy (e.g., reflectance interference spectroscopy or biolayer Interferometry) have some advantages including a relatively simple instrumentation, no need of (or straightforward) optical alignment, and low-cost biochips. However, such systems require high-resolution spectrometers, which significantly increase the cost of the final device. In addition, the simultaneous determination of more than one analyte in a sample requires the use of motorized set-ups that substantially increase the complexity of the system, the noise of the measurement, and the size and cost of the measurement apparatus thus reducing the possibility for application at the Point-of-Need.

Aiming for a small-size low cost biosensing system, an optical sensing platform based on White Light Reflectance Spectroscopy (WLRS) has been developed, where the sensor surface is a uniform layer of a silicon dioxide dielectric material on a reflective silicon surface, which under broadband illumination produces characteristic interference fringes across the visible and near infrared spectrum. The growth of the biomolecular layer on the sensor surface due to bioreaction, causes a spectral shift in the reflectance spectrum to higher wavelengths that is monitored in real time by using a dedicated optical probe (e.g., 6 illumination fibers and 1 collection fiber) and a spectrometer. By employing this principle of operation, several analytes have been quantitatively determined in a wide range of diverse applications including health, food safety, and forensics. In addition, by further advancing the WLRS method, the simultaneous determination of two analytes in the same sample has been demonstrated. However, the simultaneous determination of more than three analytes is still questionable. This limitation along with the need of a high-resolution spectrometer in the measurement set-up may present a hurdle in application of the WLRS method at the Point-of-Need.

There is therefore a need to develop systems and methods to cure the above deficiencies.

### SUMMARY

In embodiments, the techniques described herein relate to a sensor including a structured biochip including a structured film disposed on a substrate, where the structured film includes a plurality of sensing regions spatially distributed across the substrate, where the plurality of sensing regions provides spatially-varying reflectivity across the substrate, where the structured film is functionalized with binding molecules configured to selectively bind with one or more analytes; and a sample delivery sub-system including one or more channels configured to expose the structured film to a test substance; an illumination sub-system including one or more illumination sources and one or more optical elements configured to direct illumination from the one or more illumination sources to the structured film; a collection sub-system including a multi-pixel detector and one or more lenses configured to direct light from the structured film onto the multi-pixel detector in response to the illumination; and a controller including one or more processors configured to execute program instructions causing the one or more processors to receive first data from the multi-pixel detector associated with optical characteristics of the structured film prior to exposure to the test substance; receive second data from the multi-pixel detector associated with the optical characteristics of the structured film after exposure to the test substance; and determine concentrations of the one or more analytes bound to the binding molecules based on changes of the optical characteristics in the plurality of sensing regions of the structured film using the first and second data.

In embodiments, the techniques described herein relate to a sensor, where the optical characteristics include at least one of reflectivity or transmissivity characteristics.

In embodiments, the techniques described herein relate to a sensor, where the plurality of sensing regions provides a plurality of thicknesses of the structured film.

In embodiments, the techniques described herein relate to a sensor, where at least one of the plurality of sensing regions has a constant thickness.

In embodiments, the techniques described herein relate to a sensor, where at least one of the plurality of sensing regions has a graduated thickness.

In embodiments, the techniques described herein relate to a sensor, where the plurality of thicknesses of the structured film are in a range of 100 nanometers to 10 micrometers.

In embodiments, the techniques described herein relate to a sensor, where the plurality of sensing regions includes two or more sets of sensing regions, where the one or more analytes include two or more analytes, where the binding molecules in each of the two or more sets of sensing regions are different and configured to bind to different analytes of the two or more analytes.

In embodiments, the techniques described herein relate to a sensor, where the plurality of sensing regions within the two or more sets of sensing regions differ in at least one of a number of the sensing regions or physical properties of the sensing regions.

In embodiments, the techniques described herein relate to a sensor, where each of the plurality of sensing regions includes grating structures, where the grating structures in different sensing regions of the plurality of sensing regions differ in at least one of a period or a duty cycle.

In embodiments, the techniques described herein relate to a sensor, where the grating structures in at least one of the plurality of sensing regions includes elements distributed with two or more periods.

In embodiments, the techniques described herein relate to a sensor, where the grating structures in at least one of the plurality of sensing regions includes a period smaller than a wavelength in the illumination.

In embodiments, the techniques described herein relate to a sensor, where the collection sub-system includes an imaging sub-system configured to image the structured film onto the multi-pixel detector, where the first data includes one or more reference images of the structured film prior to exposure to the test substance generated with one or more spectra, where the second data includes one or more test images of the structured film after exposure to the test substance generated with the one or more spectra.

In embodiments, the techniques described herein relate to a sensor, where determining the concentrations of the one or more analytes bound to the binding molecules based on reflectivity changes in the plurality of sensing regions of the structured film using first and second images includes generating one or more difference images associated with a difference between the corresponding reference and test images associated with the one or more spectra; and determining the concentrations of the one or more analytes based on the one or more difference images.

In embodiments, the techniques described herein relate to a sensor, where the one or more spectra include one or more narrowband spectra.

In embodiments, the techniques described herein relate to a sensor, where the one or more narrowband spectra have bandwidths less than or equal to 100 nm.

In embodiments, the techniques described herein relate to a sensor, where the one or more narrowband spectra are generated directly by the one or more illumination sources.

In embodiments, the techniques described herein relate to a sensor, where the one or more illumination sources include a broadband illumination source, where the one or more narrowband spectra are generated directly by a tunable spectral filter.

In embodiments, the techniques described herein relate to a sensor, where the tunable spectral filter is located between the one or more illumination sources and the structured biochip.

In embodiments, the techniques described herein relate to a sensor, where the tunable spectral filter is located between the structured biochip and the multi-pixel detector.

In embodiments, the techniques described herein relate to a sensor, where determining the concentrations of the one or more analytes based on the one or more difference images includes determining the concentrations of the one or more analytes based on the one or more difference images using a Fourier Analysis technique.

In embodiments, the techniques described herein relate to a sensor, where determining the concentrations of the one or more analytes based on the one or more difference images includes determining the concentrations of the one or more analytes using a machine learning model trained on a training data set including difference images generated with known concentrations of the one or more analytes on the structured film.

In embodiments, the techniques described herein relate to a sensor, where the one or more spectra include a broadband spectrum.

In embodiments, the techniques described herein relate to a sensor, where the broadband spectrum has a bandwidth greater than or equal to 100 nm.

In embodiments, the techniques described herein relate to a sensor, where the illumination from the one or more illumination sources has a broadband spectrum, where the multi-pixel detector is a component of a spectrometer configured to capture reflection spectra of the plurality of sensing regions in response to the illumination, where the first data includes reference spectra of the plurality of sensing regions prior to exposure to the test substance, where the second data includes test spectra of the plurality of sensing regions after exposure to the test substance.

In embodiments, the techniques described herein relate to a sensor, where the broadband spectrum has a bandwidth greater than or equal to 100 nm.

In embodiments, the techniques described herein relate to a sensor, where the substrate includes at least one of a semiconductor, a metal, or a dielectric material.

In embodiments, the techniques described herein relate to a sensor, where the dielectric material includes at least one of silicon dioxide, silicon nitride, aluminum oxide, hafnium oxide, titanium oxide, zinc oxide, or a polymer.

In embodiments, the techniques described herein relate to a sensor, where the multi-pixel detector includes at least one of a charge-coupled-device (CCD) or a complementary metal-oxide-semiconductor (CMOS) device.

In embodiments, the techniques described herein relate to a sensor, where pixels of the multi-pixel detector are arranged in a two-dimensional array.

In embodiments, the techniques described herein relate to a sensor, where pixels of the multi-pixel detector are arranged in a one-dimensional array.

In embodiments, the techniques described herein relate to a sensor, where the sample delivery sub-system includes a microfluidic channel.

In embodiments, the techniques described herein relate to a sensor, where the microfluidic channel includes at least one of polymethyl methacrylate (PMMA) or cyclic olefin copolymer (COC).

In embodiments, the techniques described herein relate to a sensor, where a height of the microfluidic channel is greater than or equal to 100 micrometers.

In embodiments, the techniques described herein relate to a sensor, where the microfluidic channel is secured to the structured film with structured pressure sensitive adhesive.

In embodiments, the techniques described herein relate to a sensor, where the illumination from the one or more illumination sources includes wavelengths in a range of 200 nanometers to 1000 nanometers.

In embodiments, the techniques described herein relate to a sensor, where at least one of the one or more illumination sources includes a light emitting diode (LED).

In embodiments, the techniques described herein relate to a structured biochip including a structured film disposed on a substrate, where the structured film includes a plurality of sensing regions spatially distributed across the substrate, where the plurality of sensing regions provides spatially-varying optical characteristics across the substrate, where the structured film is functionalized with binding molecules configured to selectively bind with one or more analytes, where binding of the one or more analytes to the binding molecules generates an adlayer, where a thickness of the adlayer impacts the spatially-varying optical characteristics of the plurality of sensing regions, where at least one of a presence or a concentration of at least one of the one or more analytes is determinable based on the spatially-varying optical characteristics of the plurality of sensing regions.

In embodiments, the techniques described herein relate to a structured biochip, where the optical characteristics include at least one of reflectivity or transmissivity characteristics.

In embodiments, the techniques described herein relate to a structured biochip, where the plurality of sensing regions provides a plurality of thicknesses of the structured film.

In embodiments, the techniques described herein relate to a structured biochip, where at least one of the plurality of sensing regions has a constant thickness.

In embodiments, the techniques described herein relate to a structured biochip, where at least one of the plurality of sensing regions has a graduated thickness.

In embodiments, the techniques described herein relate to a structured biochip, where the plurality of thicknesses of the structured film are in a range of 100 nanometers to 10 micrometers.

In embodiments, the techniques described herein relate to a structured biochip, where the plurality of sensing regions includes two or more sets of sensing regions, where the one or more analytes include two or more analytes, where the binding molecules in each of the two or more sets of sensing regions are different and configured to bind to different analytes of the two or more analytes.

In embodiments, the techniques described herein relate to a structured biochip, where the plurality of sensing regions within the two or more sets of sensing regions differ in at least one of a number of the sensing regions or physical properties of the sensing regions.

In embodiments, the techniques described herein relate to a structured biochip, where each of the plurality of sensing regions includes grating structures, where the grating structures in different sensing regions of the plurality of sensing regions differ in at least one of a period or a duty cycle.

In embodiments, the techniques described herein relate to a structured biochip, where the grating structures in at least one of the plurality of sensing regions includes elements distributed with two or more periods.

In embodiments, the techniques described herein relate to a device including a structured biochip including a structured film disposed on a substrate, where the structured film includes a plurality of sensing regions spatially distributed across the substrate, where the plurality of sensing regions provides spatially-varying optical characteristics across the substrate, where the structured film is functionalized with binding molecules configured to selectively bind with one or more analytes, where binding of the one or more analytes to the binding molecules generates an adlayer, where a thickness of the adlayer impacts the spatially-varying optical characteristics of the plurality of sensing regions; and a sample delivery sub-system including one or more channels configured to expose the structured film to a test substance, where the one or more channels are transparent to selected wavelengths of illumination, where the where at least one of a presence or a concentration of at least one of the one or more analytes is determinable based on the spatially-varying optical characteristics of the plurality of sensing regions associated with the selected wavelengths.

In embodiments, the techniques described herein relate to a device, where the optical characteristics include at least one of reflectivity or transmissivity characteristics.

In embodiments, the techniques described herein relate to a device, where the plurality of sensing regions provides a plurality of thicknesses of the structured film.

In embodiments, the techniques described herein relate to a device, where at least one of the plurality of sensing regions has a constant thickness.

In embodiments, the techniques described herein relate to a device, where at least one of the plurality of sensing regions has a graduated thickness.

In embodiments, the techniques described herein relate to a device, where the plurality of thicknesses of the structured film are in a range of 100 nanometers to 10 micrometers.

In embodiments, the techniques described herein relate to a device, where the plurality of sensing regions includes two or more sets of sensing regions, where the one or more analytes include two or more analytes, where the binding molecules in each of the two or more sets of sensing regions are different and configured to bind to different analytes of the two or more analytes.

In embodiments, the techniques described herein relate to a device, where the plurality of sensing regions within the two or more sets of sensing regions differ in at least one of a number of the sensing regions or physical properties of the sensing regions.

In embodiments, the techniques described herein relate to a device, where each of the plurality of sensing regions includes grating structures, where the grating structures in different sensing regions of the plurality of sensing regions differ in at least one of a period or a duty cycle.

In embodiments, the techniques described herein relate to a device, where the grating structures in at least one of the plurality of sensing regions includes elements distributed with two or more periods.

It is to be understood that both the foregoing general description and the following detailed description are exemplary and explanatory only and are not necessarily restrictive of the invention as claimed. The accompanying drawings, which are incorporated in and constitute a part of the specification, illustrate embodiments of the invention and together with the general description, serve to explain the principles of the invention.

### BRIEF DESCRIPTION OF DRAWINGS

The numerous advantages of the disclosure may be better understood by those skilled in the art by reference to the accompanying figures.

FIG. 1A is a block diagram depiction of a sensor suitable for, but not limited to, label-free biosensing, in accordance with one or more embodiments of the present disclosure.

FIG. 1B is a flow diagram illustrating steps performed in a method for biosensing, in accordance with one or more embodiments of the present disclosure.

FIG. 1C is a simplified perspective view of a structured biochip having five sensing regions, where the structured film has a different height in each sensing region 106, in accordance with one or more embodiments of the present disclosure.

FIG. 1D is a simplified perspective view of a structured biochip in which the structured film includes grating structures with different characteristics in different sensing regions, in accordance with one or more embodiments of the present disclosure.

FIG. 1E is a perspective view of a sensor suitable for use with a liquid sample, in accordance with one or more embodiments of the present disclosure.

FIG. 1F is an exploded view of a sensor suitable for use with a liquid sample, in accordance with one or more embodiments of the present disclosure.

FIG. 2A is a simplified side view of a structured biochip including a reflective substrate, a structured film with varying thickness, and a functionalization layer for adhesion with an analyte, in accordance with one or more embodiments of the present disclosure.

FIG. 2B is a plot depicting signal strength as a function of position corresponding to imaging the structured biochip with narrowband illumination.

FIG. 2C is a simplified side view of an alternative sensor including a uniform layer of SiO2 on a substrate as well as a functionalization layer, in accordance with one or more embodiments of the present disclosure.

FIG. 2D is a plot of a reflection spectrum of the alternative sensor generated using a white light reflection spectroscopy (WLRS) technique, in accordance with one or more embodiments of the present disclosure.

FIG. 3A depicts a series of process steps for forming a first design of a structured biochip with a structured film having graduated thicknesses in different sensing regions, in accordance with one or more embodiments of the present disclosure.

FIG. 3B includes a plot of thickness of the structured film, in accordance with one or more embodiments of the present disclosure.

FIG. 4A is a microscope image of a structured biochip with 44 regions of linearly varying structured film thickness in water illuminated with 458±10 nm light, in accordance with one or more embodiments of the present disclosure.

FIG. 4B is an optical profilometer image of a portion of the structured film from FIG. 4A, in accordance with one or more embodiments of the present disclosure.

FIG. 4C is a plot depicting step height along the line in FIG. 4B, in accordance with one or more embodiments of the present disclosure.

FIG. 5 includes imaging and analysis data of a structured biochip during measurement and regeneration, in accordance with one or more embodiments of the present disclosure.

FIG. 6 is a plot of a dominant Fourier component from FIG. 5 as a function of time, in accordance with one or more embodiments of the present disclosure.

FIG. 7 is a simplified schematic illustrating a spectral sweep of a central frequency of a narrowband spectral filter across a wavelength range sampled by adjacent pixels of a detector, in accordance with one or more embodiments of the present disclosure.

### DETAILED DESCRIPTION

Reference will now be made in detail to the subject matter disclosed, which is illustrated in the accompanying drawings. The present disclosure has been particularly shown and described with respect to certain embodiments and specific features thereof. The embodiments set forth herein are taken to be illustrative rather than limiting. It should be readily apparent to those of ordinary skill in the art that various changes and modifications in form and detail may be made without departing from the spirit and scope of the disclosure.

Embodiments of the present disclosure are directed to systems and methods for label-free biosensing based on reflectivity variations of a structured biochip (e.g., a 3D structured biochip). For example, a structured biochip may include a structured film on a substrate. Further, the structured biochip may include various sensing regions distributed across the biochip that provide spatial variations in the reflectivity of the structured film. Such a structured biochip may be functionalized with binding molecules on which one or more analytes of interest are selectively bound. In this way, the presence and/or concentration of analytes bound to the structured film may be determined by analyzing reflectivity changes of the various sensing regions after exposure (or potential exposure) to analytes of interest. By employing this method the kinetics of bioreaction can be monitored in real-time as well. It is contemplated herein that such reflectivity changes across the sensing regions may be monitored using off the shelf and relatively low-cost illumination sources and detectors. As a result, the systems and methods disclosed herein may provide a low-cost yet accurate sensing platform.

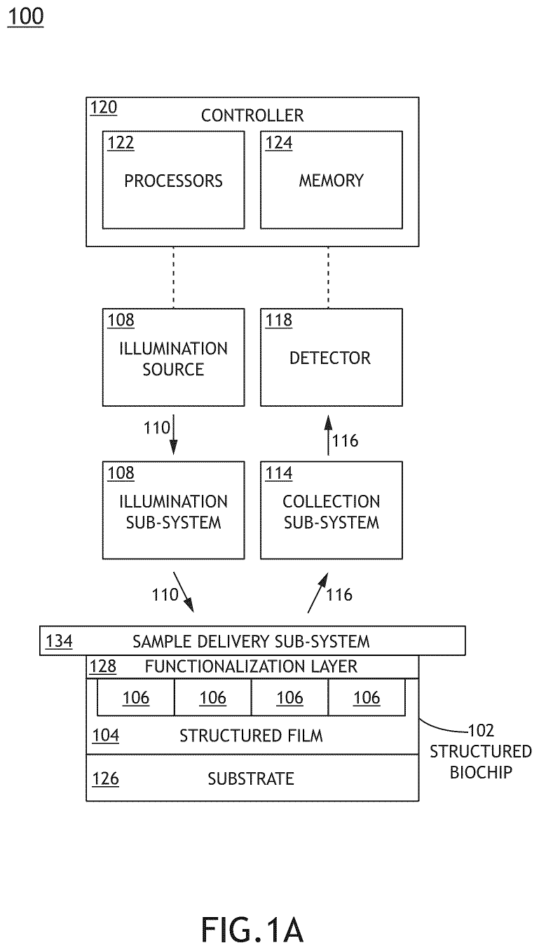

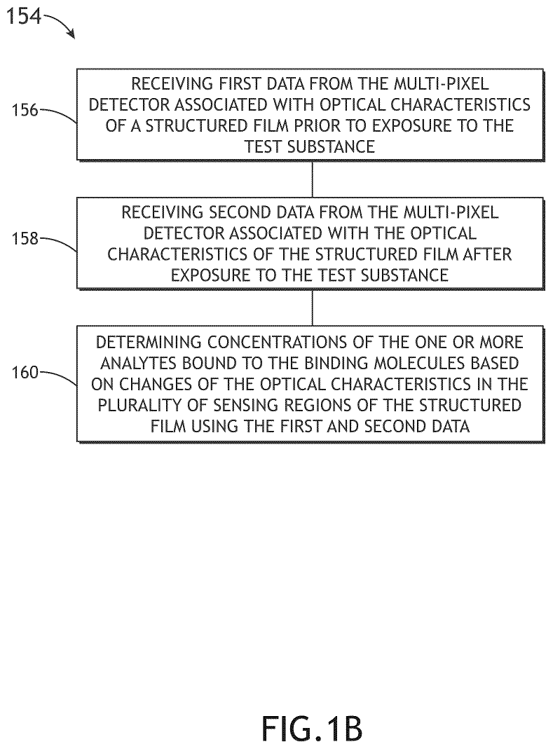

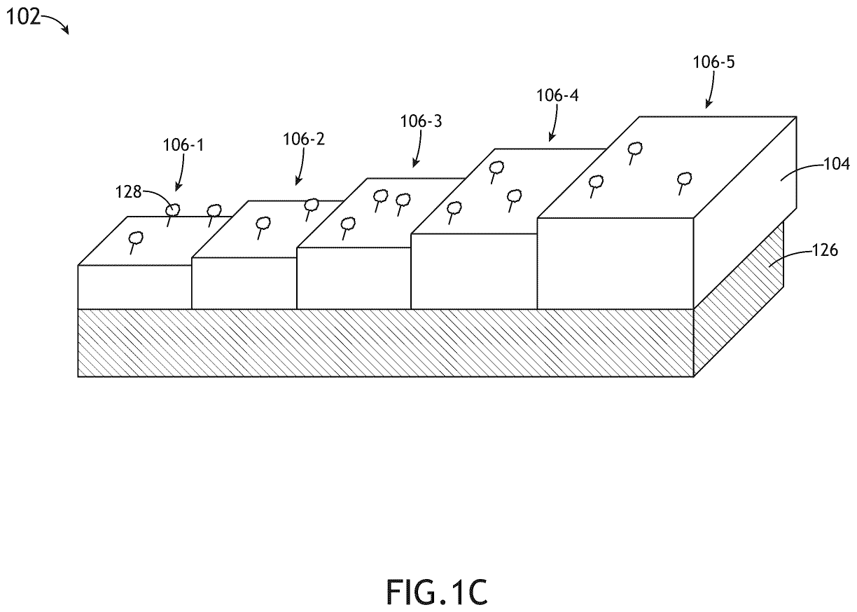

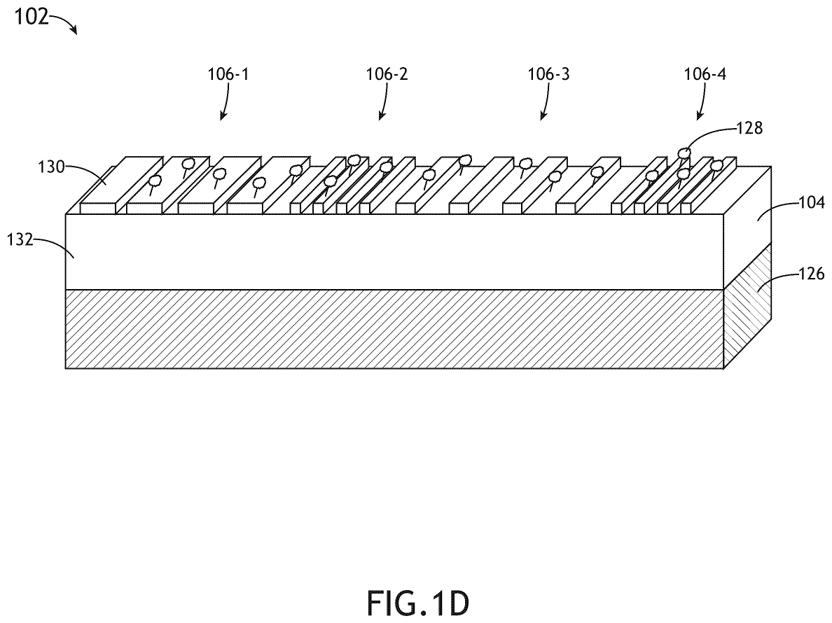

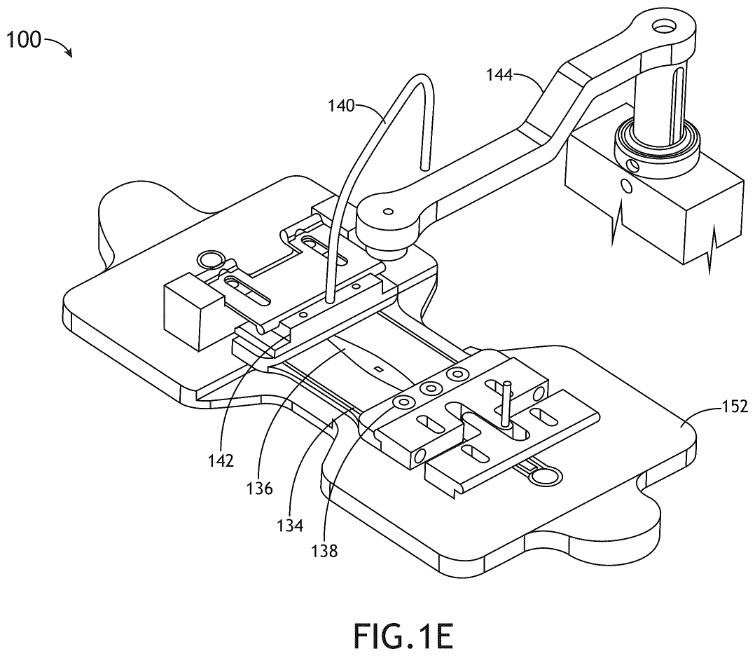

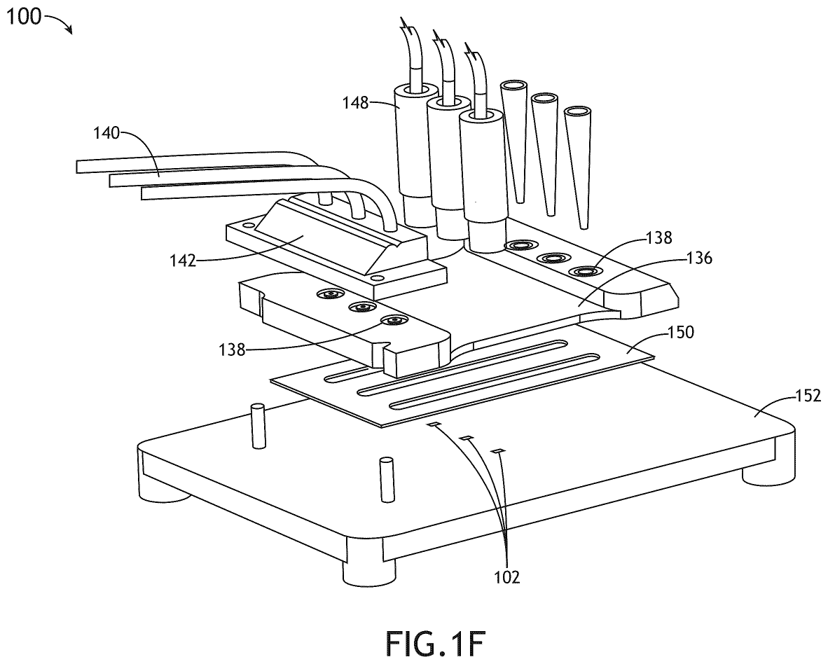

Referring now to FIGS. 1-7, systems and methods for label-free biosensing based on a structured biochip are described in greater detail, in accordance with one or more embodiments of the present disclosure.

FIG. 1A is a block diagram depiction of a sensor 100 suitable for, but not limited to, label-free biosensing, in accordance with one or more embodiments of the present disclosure.

In some embodiments, a sensor 100 includes a structured biochip 102 including a structured film 104 providing multiple sensing regions 106 having different reflectivity or transmissivity characteristics. Further, this structured film 104 is functionalized to bind to one or more analytes of interest. As a result, adhesion of an analyte to the structured film 104 across all of the sensing regions 106 result in a biomolecular adlayer which increases the thickness of the sensing regions 106 in the sensing regions 106. Further, this increase in thickness is typically uniform when the structured film 104 is uniformly functionalized. This increase in thickness may then modify the reflectivity characteristics of each sensing region 106 at one or more wavelengths.

It is contemplated herein that the systematically-varying reflectivity characteristics of the structured film 104 across the sensing regions 106 may provide robust and sensitive analyte detection, particularly when compared to a similar technique including an unstructured (e.g., flat) film. For example, the sensitivity of a reflectivity variation of a uniform film in response to analyte adhesion may be vary based on interference conditions and the presence of associated maxima or minima. In contrast, sensing regions 106 with systematically-varying reflectivity characteristics may be immune to such interference conditions since different sensing regions 106 may be impacted differently. As a result, the sensitivity of a measurement may be constant regardless of the specific thickness of the structured film 104 or adhesion layer in any particular sensing region 106. Further, sampling reflectivity variations across multiple sensing regions 106 with systematically-varying characteristics may provide increased data, which may further improve the accuracy and/or sensitivity of the measurement.

In some embodiments, the sensor 100 includes an illumination source 108 configured to generate illumination 110 and an illumination sub-system 112 to direct the illumination 110 to the structured biochip 102. In some embodiments, the sensor 100 includes a collection sub-system 114 (e.g., an imaging sub-system 114) to capture light (referred to herein as collected light 116) from the structured biochip 102 in response to the illumination 110 and direct at least a portion of this collected light 116 to a detector 118 for processing. Further, the collected light 116 may correspond to reflected light or transmitted light (e.g., a reflected portion or transmitted portion of the illumination 110). In this way, references to systematically-varying reflectivity characteristics of the sensing regions 106 are merely illustrative and should not be interpreted as limiting the present disclosure. Rather, it is contemplated that systematically-varying reflectivity characteristics may be directly correlated to systematically-varying transmissivity characteristics based at least in part on the absorption characteristics of the structured biochip 102.

In some embodiments, the sensor 100 includes a controller 120 with one or more processors 122 configured to execute program instructions stored on memory 124 (e.g., on a memory device), where the program instructions may cause the processors 122 to implement various actions. The controller 120 may be communicatively coupled to any components of the sensor 100 including, but not limited to, the detector 118. In this way, the controller 120 may receive communication (e.g., data, instructions, or the like) from any connected components and/or may direct connected components (e.g., via control signals) to perform selected actions. The controller 120 may thus directly or indirectly implement any desired actions.

The one or more processors 122 of a controller 120 may include any processor or processing element known in the art. For the purposes of the present disclosure, the term “processor” or “processing element” may be broadly defined to encompass any device having one or more processing or logic elements (e.g., one or more micro-processor devices, one or more application specific integrated circuit (ASIC) devices, one or more field programmable gate arrays (FPGAs), or one or more digital signal processors (DSPs)). In this sense, the one or more processors 122 may include any device configured to execute algorithms and/or instructions (e.g., program instructions stored in memory). In some embodiments, the one or more processors 122 may be embodied as a desktop computer, mainframe computer system, workstation, image computer, parallel processor, networked computer, or any other computer system configured to execute a program instruction as described throughout the present disclosure. Moreover, different subsystems of the sensor 100 may include a processor or logic elements suitable for carrying out at least a portion of the steps described in the present disclosure. Therefore, the above description should not be interpreted as a limitation on the embodiments of the present disclosure but merely as an illustration. Further, the steps described throughout the present disclosure may be carried out by a single controller or, alternatively, multiple controllers. Additionally, the controller 120 may include one or more controllers housed in a common housing or within multiple housings.

The memory 124 may include any storage medium known in the art suitable for storing program instructions executable by the associated one or more processors 122. For example, the memory 124 may include a non-transitory memory medium. By way of another example, the memory 124 may include, but is not limited to, a read-only memory (ROM), a random-access memory (RAM), a magnetic or optical memory device (e.g., disk), a magnetic tape, a solid-state drive and the like. It is further noted that the memory 124 may be housed in a common controller housing with the one or more processors 122. In some embodiments, the memory 124 may be located remotely with respect to the physical location of the one or more processors 122 and the controller 120. For instance, the one or more processors 122 of the controller 120 may access a remote memory (e.g., server), accessible through a network (e.g., internet, intranet and the like).

Additional aspects of the sensor 100 are now described in greater detail, in accordance with one or more embodiments of the present disclosure.

The sensor 100 may utilize any type of illumination 110 suitable for probing the spatially-varying reflectivity characteristics of the sensing regions 106. Further, the illumination source 108 may include any type of source suitable for generating a desired type of illumination 110. For example, the illumination source 108 may include, but is not limited to, a laser source, a light emitting diode (LED) source, or a lamp source.

In a general sense, the illumination 110 may have any wavelength or range of wavelengths in any spectral region including, but not limited to, ultraviolet light, visible light, or infrared light. Further, the illumination 110 may be continuous-wave or pulsed with any pulse duration.

In some embodiments, the illumination 110 is formed as narrowband illumination (e.g., illumination having a linewidth of approximately 100 nm or smaller). For example, the illumination source 108 may include a laser source with either a fixed or tunable wavelength. Such a configuration may be suitable for, but is not limited to, generating narrow-band interference patterns with the structured film 104 and/or generating discrete diffraction orders from grating structures in the structured film 104. In some embodiments, the illumination 110 is formed as broadband illumination. For example, the illumination source 108 may include a supercontinuum laser source, a white-light LED source, a halogen-tungsten light source, or the like. In some embodiments, the illumination 110 includes wavelengths in a range of 200 nanometers to 1000 nanometers.

The illumination sub-system 112 may include any type or number of optical components suitable for directing the illumination 110 to the structured biochip 102. The collection sub-system 114 may include any number or type of components suitable for collecting light (e.g., the collected light 116) from the structured biochip 102 and directing this collected light 116 to the detector 118. For example, the illumination sub-system 112 and/or the collection sub-system 114 may include one or more lenses, polarizers, spectral filters, or spatial filters. In some embodiments, the illumination sub-system 112 evenly illuminates the structured biochip 102 with the illumination 110. In some embodiments, the collection sub-system 114 images the structured biochip 102 onto the detector 118. Further, the illumination sub-system 112 and/or the collection sub-system 114 may utilize any combination of optical fibers or free-space optics to route the illumination 110 and collected light 116, respectively. In some embodiments, at least portions of the illumination sub-system 112 and the collection sub-system 114 are arranged onto a common support structure to provide compact positioning of such components near the structured biochip 102.

The detector 118 may include any type of optical detection element known in the art. For example, the detector 118 is a multi-pixel detector such as, but not limited to, a matrix of complementary metal-oxide-semiconductor (CMOS) device or a charge-coupled-device (CCD). As another example, the detector 118 includes a photodetector or an array thereof. Further, the pixels of the detector 118 may be arranged in a one-dimensional or a two-dimensional array.

Label-free biosensing based on reflectivity variations of a structured biochip 102 (e.g., a 3D structured biochip 102) is now described in greater detail, in accordance with one or more embodiments of the present disclosure.

The various sensing regions 106 of a structured biochip 102 may generally have any structures or combinations of structures suitable for providing different reflectivity characteristics within the various sensing regions 106, where variations of the reflectivity characteristics upon exposure to an analyte of interest provide qualitative or quantitative characterization of the presence and/or concentration of the analyte. Further, any particular sensing region 106 may have any suitable size. In some embodiments, sensing regions 106 have an area of at least 10 μm by 10 μm.

In some embodiments, the sensing regions 106 provide a range of thicknesses of the structured film 104. Such a configuration may provide a stepped surface. For example, the structured film 104 within each sensing region 106 may have a constant thickness, but the structured film 104 may have different thicknesses in different sensing regions 106. Further, in some embodiments, the structured film 104 is disposed on a substrate 126, which may be either reflective or transparent to the illumination 110. In such a configuration, the structured film 104 may be formed from a material that is transparent to the illumination 110 such that the reflectivity characteristics of at least some of the sensing regions 106 may be influenced by thin-film interference. For example, the structured film may be formed from a dielectric material, a polymer, or the like such as, but not limited to, silicon dioxide, silicon nitride, aluminum oxide, hafnium oxide, titanium oxide, zinc oxide, or a polymer. Further, the substrate 126 may be formed from any suitable material such as, but not limited to, a semiconductor, a metal, or a dielectric. Further, the substrate 126 may reflect or transmit the illumination 110 such that the sensing regions 106 may be characterized based on reflected or transmitted collected light 116.

As an illustration, FIG. 1C is a simplified perspective view of a structured biochip 102 having five sensing regions 106 (labeled 106-1 through 106-5), where the structured film 104 has a different height in each sensing region 106, in accordance with one or more embodiments of the present disclosure. In particular, FIG. 1C depicts a configuration in which the structured film 104 is disposed on a reflective substrate 126. Further, FIG. 1C further depicts a functionalization layer 128 suitable for binding with one or more analyzes of interest. It is to be understood, however, that FIG. 1C and the above description is merely illustrative and should not be interpreted as limiting the scope of the present disclosure. For example, FIG. 1C is not provided to scale. Further, a structured biochip 102 may generally have any number or arrangement of sensing regions 106. As an illustration, sensing regions 106 need not be arranged in order of increasing or decreasing thickness of the structured film 104. As another illustration, not all sensing regions 106 need to be disposed on a common substrate 126. In some cases, one or more sensing regions 106 are on a different substrate 126 than one or more additional sensing regions 106.

As another example, though not shown, the sensing regions 106 may provide a sloped or graduated surface to provide the varying thicknesses across the structured film.

It is contemplated herein that reflectivity of a structured film 104 having varying thicknesses across different sensing regions 106 may be determined at least in part by thin-film interference based on reflection of illumination 110 from the substrate 126 and a top surface of the structured film 104, where constructive and/or destructive interference conditions at any particular location on the structured film 104 are related to the thickness of the structured film 104 in that particular location as well as the wavelength of illumination 110. As a result, the reflectivity of a structured film 104 with varying thickness may spatially vary as a function of the thickness (e.g., a spatial interference pattern). Such a spatial reflectivity variation may generally be probed with illumination 110 at any wavelength or combination of wavelengths. For example, an image of such a structured film 104 generated with narrowband illumination 110 (e.g., typically defined as light having a bandwidth on the order of nanometers) may include a well-defined spatial interference pattern across the structured film 104 as a function of the thickness. As another example, an image of such a structured film 104 with broadband illumination 110 (e.g., white light or more generally illumination 110 having a sufficient bandwidth as to cause observably different spatial interference patterns as a function of wavelength) may include a white light interference pattern with varying spectral intensity. Such a white light interference pattern may be characterized as a superposition of narrowband spatial interference patterns.

In some embodiments, the sensing regions 106 include grating structures having different characteristics such as, but not limited to, numbers of elements, period(s) associated with a distribution of the elements, or sizes of the elements (e.g., a duty cycle or fill factor of the grating structures). In this way, each sensing region 106 may have a different reflectivity characteristic. For example, grating structures in the sensing regions 106 may include single gratings (e.g., characterized by a single period) or multiple-gratings (e.g., characterized by two or more periods). Further, the grating structures may be, but are not required to be, formed from elements that are smaller than wavelengths of illumination 110 used for a measurement (e.g., sub-wavelength structures). In this case, such grating structures may be guided mode resonance (GMR) structures that provide distinct spectral reflectivity peaks that may change based on a thickness of an adhesion layer of an analyte of interest. The reflectivity of GMR structures is generally described in R. Magnusson, et al. “Resonant Photonic Biosensors with Polarization-Based Multiparametric Discrimination in Each Channel,” Sensors (2011) 11, 1476-1488 ([20]); and “C. Zhang, et al., “High Performance of a Metal Layer-Assisted Guided-Mode Resonance Biosensor Modulated by Double-Grating,” Biosensors (2021) 11, 221 ([21]); both of which are incorporated herein by reference in their entireties.

As an illustration, FIG. 1D is a simplified perspective view of a structured biochip 102 in which the structured film 104 includes grating structures 130 with different characteristics in different sensing regions 106, in accordance with one or more embodiments of the present disclosure. In particular, FIG. 1D depicts a configuration in which the structured film 104 includes a transparent layer 132 disposed on a substrate 126 and grating structures 130 on the transparent layer 132. Further, FIG. 1D depicts a configuration with four sensing regions 106 (labeled 106-1 through 106-4), where the size and/or pitch of the grating structures 130 varies to provide different diffraction characteristics (e.g., diffraction direction, relative power in different diffraction orders, or the like) for each sensing region 106. Further, FIG. 1D depicts a functionalization layer 128 similar to that in FIG. 1C.

Referring generally to FIGS. 1C and 1D, it is contemplated herein that adhesion of an analyte to the structured film 104 may result in an adhesion layer which increases the thickness of the structured film 104 more generally. Further, this increase in thickness is typically uniform when the structured film 104 is uniformly functionalized. This increase in thickness may then modify the reflectivity of each sensing region 106 at one or more wavelengths.

As an illustration in the case of sensing regions 106 providing varying film thickness (e.g., as depicted in FIG. 1C), the presence of an adhesion layer of an analyte of interest may result in a modification of the reflectivity at each location of the structured film 104 based on the modification of the thin-film interference conditions. More generally, an increase in the thickness of the adhesion layer may result in a spatial shift of the spatial interference pattern (e.g., a phase shift) at any particular wavelength. As a result, the presence and/or concentration of the analyte of interest may be determined based on an induced change in the reflectivity of the structured film upon exposure (or potential exposure) to an analyte of interest.

As another illustration in the case of sensing regions with different grating structures (e.g., as depicted in FIG. 1D), an increase in the thickness of the adhesion layer may result in a wavelength shift of a reflective resonance peak.

The reflectivity change (or corresponding transmissivity change) of sensing regions may be measured using various techniques within the spirit and scope of the present disclosure.

In some embodiments, one or more images of the structured film 104 are generated before and after exposure (or potential exposure) to an analyte of interest, where the presence and/or concentration of the analyte is determined based on differences between these images. Such before and after images are referred to herein as reference and test images, respectively. The reference images may be generated under any conditions. In some cases, reference images are generated in the presence of a buffer. Further, the reference and test images may be generated using narrowband or broadband light.

As an illustration in the case of sensing regions 106 that provide varying thickness of the structured film, a narrowband difference image may include a periodic (e.g., sinusoidal) pixel intensity variation as a function of thickness across the various sensing regions, where an amplitude of the associated signal is related to a thickness of the adhesion layer of the analyte. This amplitude may be determined using any technique known in the art to determine the distance between the images obtained with and without the analyte adlayer, such as, but not limited to, a similarity analysis, a frequency analysis (e.g., a Fourier analysis, a cosine analysis, or the like) of the difference image, a fit of the pixel intensity to a model (e.g., least squares fit, or the like), or a machine learning model trained on difference images from the same or similar structured films with known adhesion layer thicknesses.

It is additionally contemplated herein that the systematically-varying reflectivity of the structured film 104 associated with the varying thickness across the sensing regions may provide robust and sensitive analyte detection, particularly when compared to a similar technique including an unstructured (e.g., flat) film. In particular, the use of a structured film 104 with spatially-varying thickness enables detection of a complete spatial interference pattern before and after exposure (or potential exposure) to an analyte of interest and maintains a constant sensitivity regardless of the adhesion layer thickness. In contrast, measurements based on the reflectivity variations of a single film of constant thickness when exposed to an analyte are limited to a single intensity variation associated with a variation of thin-film interference. However, since interference signals are sinusoidal, the sensitivity of such a measurement may diminish for conditions associated with a maximum or minimum of the interference signal.

Referring now to FIGS. 1A, 1E, and 1F, techniques for bringing a sample with potential analytes into contact with at least one structured biochip 102 are described in greater detail, in accordance with one or more embodiments of the present disclosure.

In some embodiments, the sensor 100 includes a sample delivery sub-system 134 suitable for exposing the structured biochip 102 (e.g., the structured film 104 on the structured biochip 102) to a test substance. The sample delivery sub-system 134 may include any number or type of components suitable for delivering a sample that may potentially include one or more analytes of interest to a structured biochip 102, where the sample may be in a liquid phase or a gas phase. For example, the sample delivery sub-system 134 may include one or more channels for the delivery of the sample such as, but not limited to fluidic channels, microfluidic channels, gas channels, tubes, manifolds, or the like. As another example, the sample delivery sub-system 134 may include one or more pumps (e.g., vacuum pumps, or the like) to control the flow of the sample by the structured biochip 102. As another example, the sample delivery sub-system 134 may include various inlet and outlet ports, sample storage containers, or the like.

FIGS. 1E and 1F depict a non-limiting configuration of a sensor 100 including a sample delivery sub-system 134.

FIG. 1E is a perspective view of a sensor 100 suitable for use with a liquid sample, in accordance with one or more embodiments of the present disclosure. In particular, FIG. 1E depicts a configuration in which the sample delivery sub-system 134 includes a microfluidic channel 136 to expose the structured biochip 102 to a test liquid. The microfluidic channel 136 may formed from any material suitable for containing the sample that is also transparent to the illumination 110 such as, but not limited to, polymethyl methacrylate (PMMA) or cyclic olefin copolymer (COC). In some embodiments, the microfluidic channel 136 is perforated with conical holes 138 (e.g., two holes) for the supply and/or return of reagents and for the drain. FIG. 1E further depicts tubing 140 attached to a manifold 142 on a return side of the microfluidic channel 136, which may connect to a pump (not shown). FIG. 1E also depicts a support structure 144 for positioning portions of the illumination sub-system 112 and/or the collection sub-system 114 to provide interrogation of the structured biochip 102 through the microfluidic channel 136.

FIG. 1F is an exploded view of a sensor 100 suitable for use with a liquid sample, in accordance with one or more embodiments of the present disclosure. The configuration of the sensor 100 in FIG. 1F is substantially similar to that depicted in FIG. 1E, except that the sensor 100 includes three microfluidic channels 136, each providing characterization of a different structured biochip 102, along with corresponding tubing 140. FIG. 1F further depicts nozzles 146 to provide sample injection separate support structures 148 for portions of the illumination sub-system 112 and collection sub-system 114 suitable for characterizing each of the structured biochips 102 in parallel.

The height of the microfluidic channel 136 may be, but is not required to be, secured by structured PSA 150 (Pressure Sensitive Adhesive) of height of at least 100 μm to screen out the interference from the liquid. Further, the microfluidic channel 136 may be directly attached to the structured biochip 102 or may be attached to a separate mechanism and brought into proximity with the structured biochip 102. For example, FIGS. 1E and 1F depict a flat plate 152 to interface with the PSA 150 and form a bottom of the microfluidic channel 136. Such a plate 152 may further include a recess to secure a structured biochip 102.

As another example, though not shown, the sample delivery sub-system 134 may include a channel and associated nozzles for exposing the structured film to a gaseous test substance. Accordingly, it is to be understood that any examples or descriptions herein related to fluidic testing of liquids are merely illustrative and should not be interpreted as limiting.

Further, it is contemplated herein that any combination of components of the sensor 100 may be provided as a disposable element. As an illustration, in some embodiments, a microfluidic channel 136 and associated components suitable for flowing a sample across a structured biochip 102 (e.g., the PSA 150, the flat plate 152, the tubing 140, manifolds 142, or the like). In some embodiments, a package including one or more structured biochips 102 along with one or more microfluidic channels 136 and associated components suitable for flowing a sample across the one or more structured biochips 102 are provided as a disposable element.

Referring now to FIG. 1B, FIG. 1B is a flow diagram illustrating steps performed in a method 154 for biosensing, in accordance with one or more embodiments of the present disclosure. Applicant notes that the embodiments and enabling technologies described previously herein in the context of the sensor 100 should be interpreted to extend to the method 154. It is further noted, however, that the method 154 is not limited to the architecture of sensor 100.

In some embodiments, the method 154 includes a step 156 of receiving first data from the multi-pixel detector 118 associated with optical characteristics of a structured film 104 prior to exposure to the test substance. In some embodiments, the method 154 includes a step 158 of receiving second data from the multi-pixel detector 118 associated with the optical characteristics of the structured film 104 after exposure to the test substance. The optical characteristics associated with the first data and second data may correspond to any combination of reflectivity characteristics or transmissivity characteristics. In some embodiments, the method 154 includes a step 160 of determining concentrations of the one or more analytes bound to the binding molecules based on changes of the optical characteristics in the plurality of sensing regions 106 of the structured film using the first and second data. The step 160 may utilize any techniques or combinations of techniques suitable for characterizing the changes of the optical characteristics of the sensing regions 106. For example, the step 160 may include generating one or more difference images associated with a difference between the corresponding reference and test images associated with the one or more spectra, and determining the concentrations of the one or more analytes based on the one or more difference images.

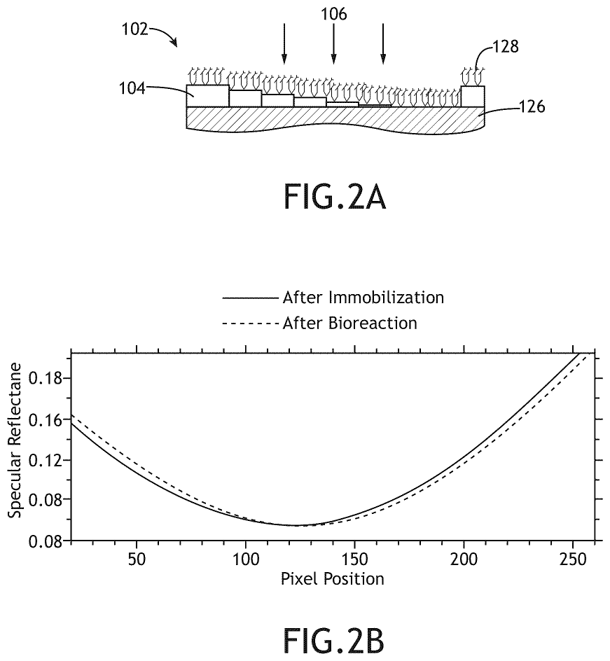

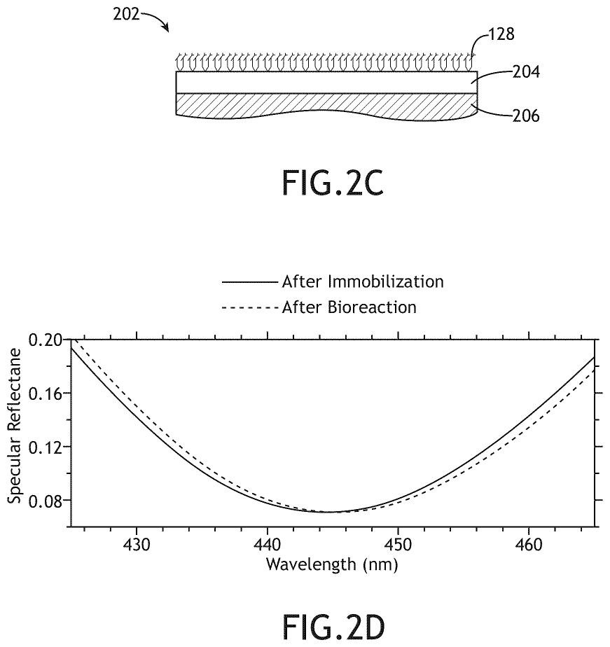

Referring now to FIGS. 2-6, a non-limiting example of a sensor 100 as disclosed herein in which a structured biochip 102 includes a structured film 104 with different thicknesses in different sensing regions 106. In FIGS. 2-6, a novel optical label-free biosensing approach is introduced aiming to remove the current limitations of optical biosensors in their application of on-site food analysis. The proposed methodology (GRADual thin film IntErferometry, GRADE) is based on light interferometry employing an illumination source 108 of a few nm spectral width and a 3D micro-patterned structured biochip 102 consisting of a structured film 104 having multilevel staircase structures, fabricated through grayscale electron beam lithography and appropriate pattern transfer on thick dielectric layer. The formation of the biomolecular adlayer on the structured biochip 102 is monitored with a 2D camera detector 118, without the need for broadband illumination sources and spectral analysis instrumentation. Consequently, this novel sensing approach has the potential to enable the cost-efficient monitoring of bioreactions employing a simple optical setup, a low-cost photodetector and light source, thus being an ideal platform for use at the point-of-need. In this non-limiting example, the sensor 100 is demonstrated for the detection of aflatoxin B1 (AFB₁), a very potent carcinogen detected in various food categories.

Spectral interferometric techniques may be used to monitor and analyze the reflectance interference fringes created upon interaction of light with the structured biochip 102 to detect changes in the length of the optical path caused by specific bioreactions occurring between binding molecules immobilized onto the surface and complementary biomolecules in the sample. The optical immunosensor (GRADual thin film IntErferometry, GRADE) is based on interferometry of reflected narrow band light from a 3D structured layer of SiO₂ of spatially varying thickness (the structured film 104) on top of a Si substrate 126, where the bioreactions take place. Since the transparent SiO₂ structured film 104 is not planar but consists of multiple regions with different thickness, the corresponding reflected light intensity of each one of them is different, thus creating an “interference” pattern depending on the designed surface thickness profile that can be recorded from a multi-pixel detector 118.

FIGS. 2A-2D contrast measurements based on a sensor 100 as disclosed herein with an alternative device including a uniform thin film. FIG. 2A is a simplified side view of a structured biochip 102 including a reflective substrate 126, a structured film 104 with varying thickness (here, formed from SiO₂), and a functionalization layer 128 for adhesion with an analyte, in accordance with one or more embodiments of the present disclosure. FIG. 2B is a plot depicting signal strength as a function of position corresponding to imaging the structured biochip 102 with narrowband illumination.

FIG. 2C is a simplified side view of an alternative sensor 202 including a uniform layer 204 of SiO₂ on a substrate 206 as well as a functionalization layer 208, in accordance with one or more embodiments of the present disclosure. FIG. 2D is a plot of a reflection spectrum of the alternative sensor 202 generated using a white light reflection spectroscopy (WLRS) technique, in accordance with one or more embodiments of the present disclosure.

In the example depicted in FIGS. 2C-2D, the thickness of the uniform SiO2 layer 204 corresponds to one minimum in the 400-700 nm spectral range, which shifts to higher wavelengths due to the bioreaction providing the means for its real time monitoring with the WLRS set-up. By careful design (i.e., by adjusting the thickness range) of the 3D structured SiO2 structured film 104 in the structured biochip 102 shown in FIG. 2A, the number of extrema (minima and maxima) displayed in the CCD can be tuned, as shown in FIG. 2B, to obtain a reflectance intensity profile along the surface for monochromatic light, which may be similar with the WLRS spectral interference pattern. When a biomolecular layer built up on top of the dielectric film in both methodologies (WLRS and GRADE), the optical path changes and a shift is detected. However, despite the similarity, the two methods have different detection principles. In the WLRS methodology, a spectral shift is measured, while in the GRADE methodology utilizing the structured biochip 102 as disclosed herein, reflectance variation is observed by a pixel shift. In this way, FIGS. 2A-2D demonstrate that the use of a structured biochip 102 as disclosed herein may achieve detection comparable to existing techniques using simpler, less expensive components. In particular, the use of a structured biochip 102 as disclosed herein does not require a spectrometer to directly measure a spectrum of collected light and may rather use a simple monochrome imaging detector 118.

However, it is to be understood that FIGS. 2A-2D are provided solely for illustrative purposes and should not be interpreted as limiting the scope of the present disclosure. For example, as described above, the design of the structured biochip 102 was configured to provide a signal that was easily comparable with a reflection signal from the additional sensor 202 using the WLRS technique. However, this is not a limitation and the structured biochip 102 may provide a structured film 104 with any suitable design.

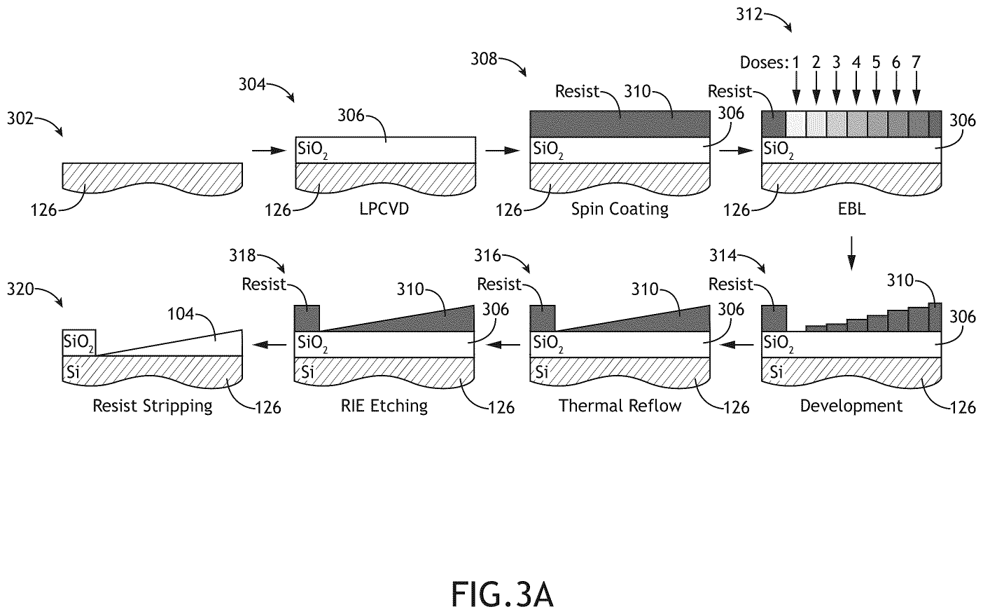

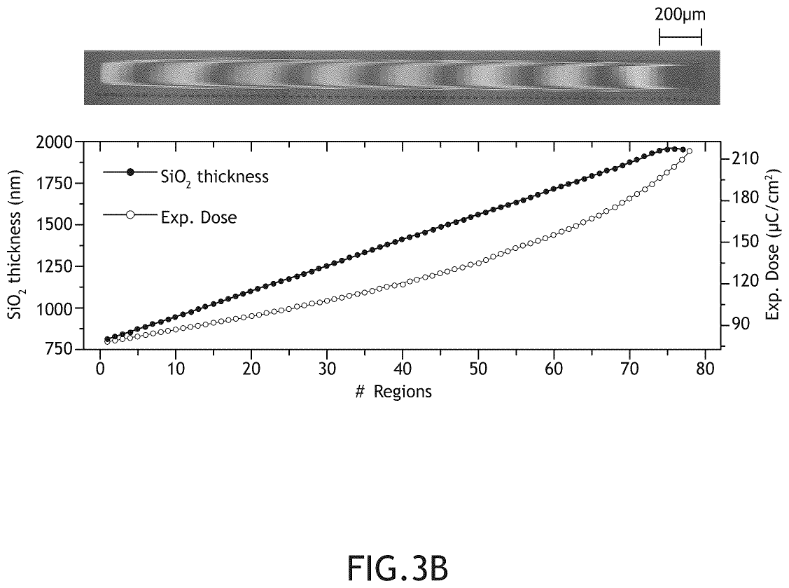

FIG. 3A depicts a series of process steps for forming a first design of a structured biochip 102 with a structured film 104 having graduated thicknesses in different sensing regions 106, in accordance with one or more embodiments of the present disclosure.

For the realization of the 3D structured biochip 102, a fabrication flow-chart was developed, based on electron beam lithography (EBL), via careful local tuning of the exposure dose, according to the characteristic contrast curve of the resist employed. In order to analyze the reflection signal by as many pixels on the detector 118 as possible, grayscale EBL was employed. This technique allowed for tailor-made pattern transfer and fabrication of multiple sensor surfaces, each one designed to assume a 3-dimensional topography on 3-μm thick thermally grown SiO₂ structured film 104 on top of a Si substrate 126, with thickness difference between adjacent regions of the structured film 104 on the order of 15 nm.

Panel 302 depicts a silicon substrate 126 after cleaning in a Piranha solution, dehydration and coating with HMDS (hexamethyl disilazane) to improve resist adhesion. Panel 304 depicts the deposition of a film 306 of SiO2 (e.g., a film to be patterned to form the structured film 104). For example, panel 304 depicts the use of a using a low-pressure chemical vapor deposition (LPCVD) process. However, this is merely illustrative and should not be interpreted as limiting the scope of the present disclosure. Any suitable process may be utilized including, but not limited to, thermal oxidation, LPCVD, plasma-enhanced chemical vapor deposition (PECVD), or sputtering. Panel 308 depicts the deposition of a resist 310 over the film 306 using a spin coating process. For example, SiO₂/Si wafers with were spin coated with negative tone resist at 1000 rpm and subsequently baked at 85° C. on a hot plate for 65 s. Panel 312 depicts graduated exposure of the resist 310. For example, dose-modulated e-beam lithographic (EBL) exposure was performed using an e-beam writer operating at 100 kV, with a beam current set to 7 nA. However, this is merely illustrative and should not be interpreted as limiting the scope of the present disclosure. Any suitable process may be utilized including, but not limited to, I-line lithography, deep ultraviolet (DUV) lithography, gray-scale lithography, or nano-imprint lithography (NIL). Panel 314 depicts development of the structures for 120 seconds by immersion and gentle stirring in 0.16 N TMAH (tetramethylammonium hydroxide) aqueous solution, at room temperature. For the given process parameters, the exposed resist 310 led to low contrast values (γ), typically equal to 1.14±0.1. The exposure dose matrix, ranged from 78 to 184 C/cm² and was carefully sequenced to exploit the locally non-uniform development rates observed during the development process, in order to achieve an even height step difference, as illustrated in FIG. 3.

Panel 316 depicts a thermal reflow step to modify the shape of the “binary” resist 310 and smoothen the surface. For example, reflow was performed at 150° C. (Treflow, above the resist's T_{g}) for 5 min to smoothen the surface. Panel 318 depicts a step of transferring the pattern in the resist 310 to the film 306. For example, the resist 310 was transferred into the SiO₂ film 306 using a standard reactive ion etching (RIE) process at an etch rate of 28 nm min⁻¹ in SiO₂ and 15 nm min⁻¹ in AR-N. Panel 320 depicts a structured biochip 102 after a resist stripping step, where the film 306 is now the structured film 104.

After resist stripping, each structured biochip 102 (sensor surface) was diced and prepared for functionalization and encapsulation with a dedicated microfluidic cell.

FIG. 3B includes a plot of thickness of the structured film 104, in accordance with one or more embodiments of the present disclosure. The x-axis represents region number (e.g., regions of different exposure dose in panel 312) and the y-axis represents thickness of the structured film 104 formed from SiO₂ in this example. The inset 322 depicts an interferogram of the structured film 104 to visually depict the graduated thickness variation.

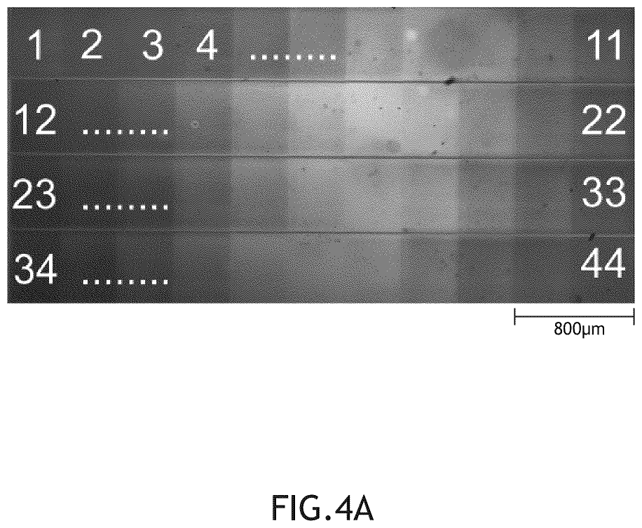

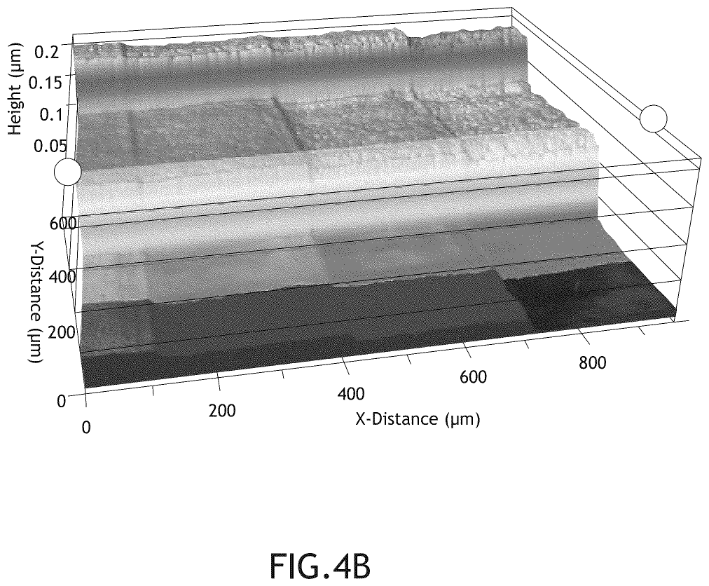

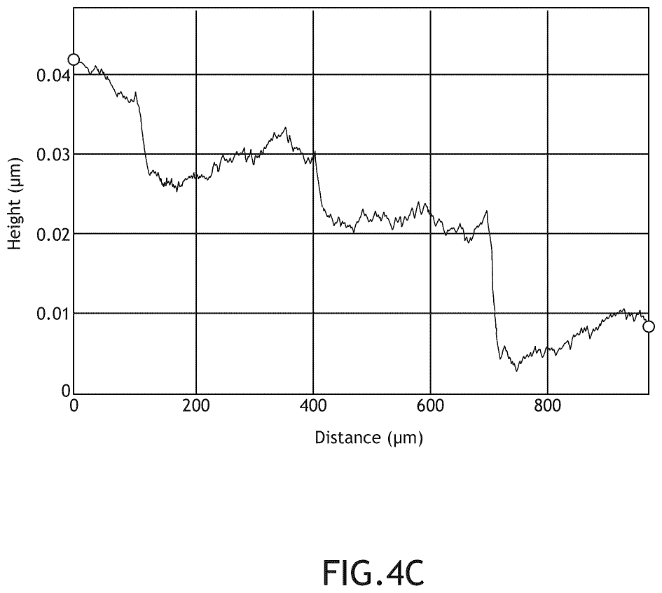

FIGS. 4A-4C illustrate an alternative design of a structured film 104 on a structured biochip 102. In particular, FIGS. 4A-4C depict a configuration in which sensing regions 106 are provided as square regions i on a raster 11×4 arrangement, each one with dimensions of 300 μm×400 μm, which is designed to fill the entire field of view of the selected detector 118. It is noted that such a design may be fabricated using steps similar to those shown in FIG. 3A, except that the exposure, development, and/or reflow steps (e.g., panels 312-316).

FIG. 4A is a microscope image of a structured biochip 102 with 44 regions of linearly varying structured film 104 thickness in water illuminated with 458±10 nm light, in accordance with one or more embodiments of the present disclosure. The various sensing regions 106 are labeled as overlays on the image as an index i(1-44), where the thickness values varied as dᵢ≈1000 (nm)+15 (nm)×i.

FIG. 4B is an optical profilometer image of a portion of the structured film 104 from FIG. 4A, in accordance with one or more embodiments of the present disclosure. In particular, FIG. 4B depicts how the area index changes sequentially along the small steps, where three of the four rows of the sensor can be distinguished. FIG. 4C is a plot depicting step height along the line 402 in FIG. 4B, in accordance with one or more embodiments of the present disclosure.

The optical setup (e.g., the illumination sub-system 112 and the collection sub-system 114) for bioreaction monitoring in this example was based on a microscope using a ×4 objective lens, a halogen light illumination source 108 with a 458±10 nm bandpass filter (that can be easily fitted on any microscope), and a CMOS detector 118 for the imaging. The thickness difference of two adjacent regions (around 15 nm) in water results in approximately 1% difference in reflectivity at 458 nm wavelength, which provides enough visual contrast and can be clearly distinguished from the microscope image of FIG. 4A.

The biochips 102 were first cleaned with acetone and 2-propanol, and then hydrophilized and further cleaned by immersion in a Piranha solution (1:1 H₂SO₄/H₂O₂, 30% v/v) for 20 min. After washing with distilled water and drying with N₂, the chips were immersed in a 2% (v/v) aqueous (3-aminopropyl)triethoxysilane (APTES) solution for 20 min. Finally, they were washed again with distilled water and dried with N₂ and then thermally cured for 20 min at 120° C. The APTES-functionalized chips were left for at least 48 h at room temperature prior to spotting with a 50 μg/mL AFB₁-bovine serum albumin conjugate solution (AFB₁-BSA; Aokin AG, Germany) in 0.05 M carbonate buffer, pH 9.2, using the BioOdyssey Calligrapher Mini Arrayer (Bio-Rad Laboratories, Inc.). During spotting, the humidity was set at 75% and the temperature at 15° C. to avoid drying of the deposited solution, whereas after spotting the chips were washed with 10 mM Tris-HCl, pH 8.25, containing 9 g/L NaCl, and blocked through immersion for 1 h in 10 mg/mL BSA solution in 0.1 M NaHCO₃, pH 8.5. Following that, the surfaces were washed with distilled water, dried under N₂ flow and used for the assay.

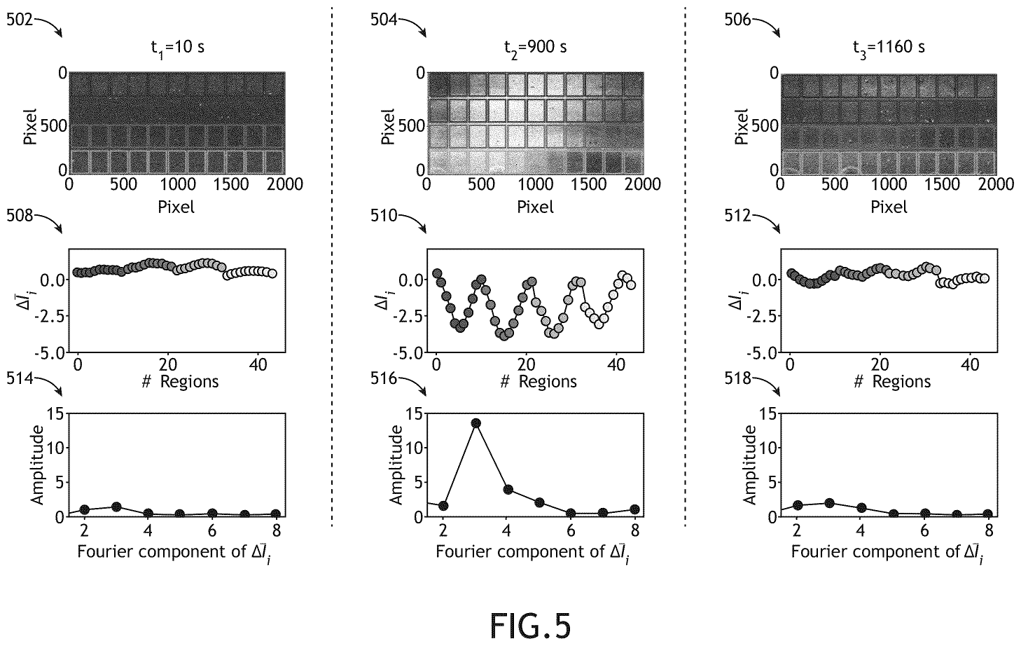

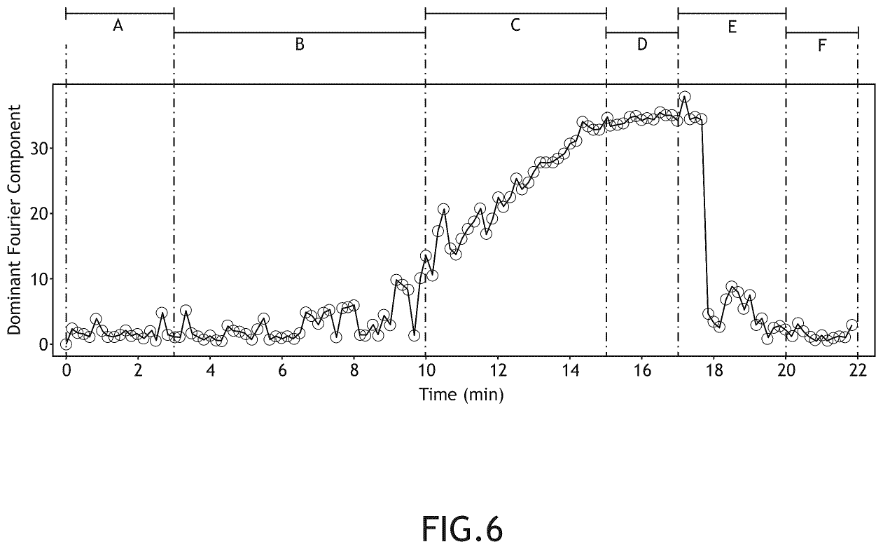

FIGS. 5 and 6 depict data associated with exposure of a structured biochip 102 to an analyte and subsequent regeneration of the structured biochip 102, which demonstrates both the use of the structured biochip 102 for accurate sensing and the ability to reuse such a structured biochip 102 for multiple tests.

Each structured biochip 102 was then assembled with a microfluidic channel 136, placed on a docking station and equilibrated with assay buffer (50 mM Tris-HCl, pH 7.8, 9 g/L NaCl, 5 g/L BSA, 0.5 g/L NaN3) for 3 min, prior to running 1:1 (v/v) mixtures of AFB1 calibrators with a 1 μg/mL of anti-AFB1 antibody (Aokin AG, Germany) solution in assay buffer for 7 min at a flow rate of 40 μL/min. Following that, a 10 μg/mL goat anti-mouse IgG antibody solution in assay buffer was flowed for 5 min. Finally, the structured biochip 102 was washed 2 min with assay buffer and regenerated by passing 0.5% (w/v) SDS solution, adjusted at pH 1.3 with 0.1 M HCl, for 3 min, and then equilibrated again with assay buffer. In all steps, the solutions were run at a flow rate of 40 μL/min. Prior to the next measurement, the structured biochip 102 was equilibrated with assay buffer.

The reflectivity of the surface of each sensing region 106 is expected to change due to the formation of the biomolecular adlayer on the functionalization layer 128, where the total thickness of the biomolecular adlayer may be, but is not required to be, of the order of 1 nm. The signal of the detector 118 pixel p, I(p, a(t),λ) depends on the local thickness of the imaged sensing region 106, the adlayer thickness a(t) which varies during the measurement, and the wavelength A. The presence of an adlayer with varying thickness can be detected from the change in the reflected intensity recorder by the detector 118 during the measurement:

$$Δ I ( p , a ( t ) , λ ) = I ( p , a ( t ) , λ ) - I ( p , a ( 0 ) , λ ) \qquad ( 1 )$$

where a(0) is the adlayer thickness in the beginning of the measurement. From the images obtained we assign an average intensity in each rectangular region, which includes Nᵢ pixels:

$${\overline{Δ I}}_{i} = \frac{1}{{N}_{i}} {\sum }_{p ∈ i} Δ I ( p , a ( t ) , λ ) \qquad ( 2 )$$

to compensate for contrast variations due to bubbling or other image artifacts due to the flow during the experiment.

FIG. 5 includes imaging and analysis data of a structured biochip 102 during measurement and regeneration, in accordance with one or more embodiments of the present disclosure. In particular, FIG. 5 includes data at times t₁=10 s, t₂=−900 s and t₃=1 160 s, where t₁ corresponds to the beginning of the assay when the assay buffer was run over the surface of the structured biochip 102, t₂ corresponds to reaction with the secondary antibody solution, and t₃ corresponds to the end of the assay when the surface of the structured biochip 102 was regenerated to remove all bound antibodies.

The progress of the assay was monitored by acquiring images of the structured biochip 102 every 10 s. Panels 502-506 depict difference microscope images (difference ΔI) for images at the respective times, panels 508-512 depict plots of average difference intensity Δ̅I̅ᵢ̅ for the sensing regions 106 at the respective times, and panels 514-518 depict plots of Fourier Amplitude of various Fourier components of the signals in panels 508-512.

When the adlayer thickness increases, one can clearly observe a sinusoidal variation of Δ̅I̅ᵢ̅ with the thickness of the SiO₂ structured film 104. For t₂=900 s, at least 4 periods can be clearly distinguished so that the variation of the reflectivity due to the adlayer which allows for an accurate determination of the amplitude of the variation by calculating the Fourier transform of Δ̅I̅ᵢ̅ versus SiO₂ structured film 104 thickness, which is expected to be proportional to the thickness of the adlayer. The magnitude of the dominant Fourier component correlates directly and quantifies the adlayer thickness, where low image contrast (t₁=10 s and t₃=1160 s) suggests low adlayer thickness, while when the contrast is high (t₂=900 s) the adlayer thickness increases.

This procedure allows one to monitor the progress of the assay. FIG. 6 is a plot of a dominant Fourier component from FIG. 5 (e.g., in panels 514-518) as a function of time, in accordance with one or more embodiments of the present disclosure. In particular, FIG. 6 demonstrates the evolution of the dominant Fourier component upon running a zero AFB₁ calibrator solution. Specifically, the signal recorded during step A where the structured biochip 102 is equilibrated with assay buffer does not detect any difference attributed to the formation of biomolecular adlayer as expected, while at step B, where the reaction of the specific anti-AFB₁ antibody with the immobilized onto the structured biochip 102 AFB₁-protein conjugate takes place, a change can be sensed at t=6.5 min, 2 min after the start of introducing the anti-AFB₁ antibody on the 3D structured surface. The binding of the specific anti-AFB₁ antibody (primary antibody) can be detected up to the time the secondary antibody is introduced at step C (from 10 to 15 min), with a signal fluctuation at 8-9 min, which is attributed to the very small thickness (around 0.2 nm) of the effective biomolecular layer corresponding to step B. The reaction with the secondary antibody, at step C, resulted in significant signal enhancement due to the binding of multiple secondary antibodies per molecule of surface bound specific antibody, while the signal is stable when assay buffer was run again over the chip at step D. Introduction of regeneration solution caused an abrupt decrease of the signal due to removal of bound primary and secondary antibody (step E) and equilibration with assay buffer stabilized the signal at a value close to the signal prior to assay (step F).

The signal obtained for the zero AFB₁ calibrator, depicted in FIG. 6, is the maximum signal obtained for this assay since a competitive immunoassay format is followed. In this assay format, detection sensitivity depends on the specific antibody binding affinity and the variation of the signal corresponding to zero calibrator. Thus, although a full calibration curve was not received, based on the performance of the same antibody in the WLRS sensor (e.g., as depicted in FIG. 2B) and the similar variation of values received for repetitive measurements of the zero calibrator with the two sensors (<3.5%), it is expected that the assay detection limit would be lower than 0.1 ng/mL. Regarding the specificity of the detection based on previous studies with the same antibody, it is anticipated that not significant cross-reactivity against AFB₂ or the major mycotoxins of the fumonisins and trichothecenes group are expected.

Referring again generally to FIGS. 1-6, the systems and methods disclosed herein provide an alternative approach of the WLRS methodology for label-free detection of bioreactions. The systems and methods disclosed herein rely on a simple and cost-effective set-up in contrast with other optical label-free detection techniques. For instance, the illumination 110 does not require broadband illumination source 108 source but may instead include relatively more cost-effective solutions such as, but not limited to, LEDs or spectral filters. Further, the signal may be recorded by a camera detector 118 rather than a spectrometer. Finally, the fabrication of the biochips 102 can be performed by cost-effective high-volume manufacturing employing UV or nanoimprint lithography instead of EBL. The illustrative but non-limiting results herein on the detection of aflatoxin B1 are in a good agreement with similar study using the WLRS methodology supporting the potential impact of the novel detection principle in application fields such as the food industry, the pharmaceutical industry and the bio diagnostics where on-site determinations are of utmost importance.

Various additional techniques are now described, in accordance with one or more embodiments of the present disclosure.

In some embodiments, the spectral reflection properties of the structured film 104 are measured.

In some embodiments, multiple sets of narrowband images of the structured film 104 are generated using different narrowband spectra. Such a technique may be characterized as hyperspectral imaging and may provide additional data to further increase the sensitivity and/or robustness of the measurement. The different narrowband spectra may be generated using any technique known in the art. For example, a sensor 100 may include multiple narrowband illumination sources 108 configured to generate light with different narrowband spectra. As another example, a sensor may include a broadband illumination source and a tunable spectral filter to generate different narrowband spectra. Further, such a tunable spectral filter may be placed at any suitable location such as, but not limited to, between the illumination source 108 and the structured biochip 102 or between the structured biochip 102 and a detector 118 (e.g., a multi-pixel imaging sensor). Any type of tunable spectral filter may be used. In some embodiments, the tunable spectral filter is a micro-electro-mechanical system (MEMS) device such as, but not limited to, a tunable Fabry-Perot cavity filter in which a central passed wavelength is controlled (e.g., via the controller 120) based on a position of a mirror in the cavity. In some embodiments, the tunable spectral filter includes an angle-sensitive dielectric filter in which a central passed wavelength is controllable based on an angle of the filter with respect to incident light.

In some embodiments, a sensor 100 includes a dispersive element (e.g., a grating, a prism, or the like) to spatially disperse the spectrum of collected light 116 from a structured film in response to broadband illumination 110. For example, the sensor 100 may include an imaging spectrometer in which the structured film 104 (or a portion thereof) is spatially resolved on a multi-pixel detector 118, where the spectrum is distributed along one direction on an associated sensing element (e.g., across multiple pixels). As an illustration, the sensing regions may be distributed along one direction and imaged on a multi-pixel detector 118, where a dispersive element disperses light from each sensing region 106 of the structured biochip 102 along an orthogonal direction on the multi-pixel detector 118. As another illustration, the sensing regions 106 may be distributed in two dimensions (e.g., in an array, a random pattern, in a semi-random pattern, or the like). In this case, the sensing regions 106 may be sufficiently separated or sufficiently large that the spectrum from each sensing region may be uniquely detectable on the multi-pixel detector 118.

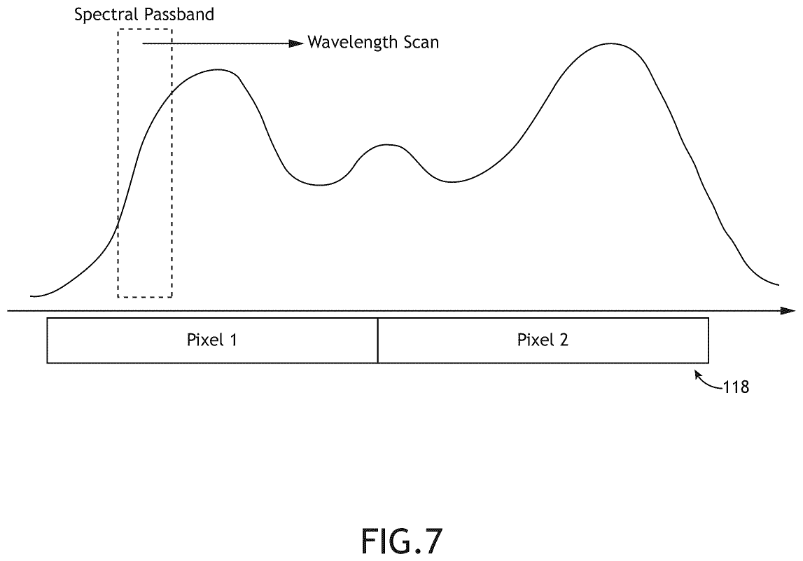

In some embodiments, the sensor 100 includes a tunable spectral filter (e.g., prior to the sensor) in applications where a spectrum from the structured film is spatially dispersed on the detector 118 (e.g., across multiple pixels). Such a configuration may enhance the ability to resolve or characterize the spectral reflectivity of the structured film in response to an analyte and thus improve the sensitivity and/or accuracy of the sensing technique. In particular, this technique may improve the resolution that is otherwise limited by the pixel size. For example, the pixels of a sensitive CMOS detector 118 are of typically in a range of 1 to 16 micrometers, where larger pixel size generally collect more light. However, increasing the pixel size decreases the spatial sampling resolution and may thus reduce performance metrics such as, but not limited to, the sensitivity, dynamic range, or a combination thereof. In some embodiments, a tunable spectral filter is used to scan a narrow spectral passband of light transmitted to each pixel. In particular, the spectral passband may be selected to be narrower than a bandwidth sampled by each pixel (e.g., as determined by the spatial dispersion of the reflected light across the pixels). FIG. 7 is a simplified schematic illustrating a spectral sweep of a central frequency of a narrowband spectral filter across a wavelength range sampled by adjacent pixels of a detector 118, in accordance with one or more embodiments of the present disclosure.

Scanning the central wavelength of the tunable spectral filter and capturing associated data may thus enable measurements of the reflection spectrum from the structured film than otherwise provided by the pixel size. Further, correlating the signals between adjacent pixels, one can create a “superresolution” pixel that has enhanced information compared to two larger adjacent pixels.

Referring now generally to FIGS. 1-7, various techniques for sensing multiple analytes (e.g., multiplexing) are described in greater detail, in accordance with one or more embodiments of the present disclosure.

In some embodiments, the structured film 104 may include multiple sets of sensing regions 106 that are functionalized with different binding molecules that selectively bind to different analytes. More broadly, the different sets of sensing regions 106 may be considered separate biochips 102 or sub-sensors of a common multiplexed structured biochip 102. For convenience, each set of sensing regions 106 is referred to herein as a separate structured biochip 102, though it is to be understood that this does not imply or limit the characteristics of the device. For example, many biochips 102 may share a common substrate 126. In some cases, a structured biochip 102 may be considered as having multiple sets of sensing regions 106.

Further, the properties and number of the sensing regions 106 within each structured biochip 102 may be the same or may differ in ways suitable for detection of the different analytes. In a general sense, any number of biochips 102 may be fabricated on a common substrate 126 such as, but not limited to, 2, 10, 20, or greater. For example, it may be the case that a set of sensing regions 106 suitable for a particular analyte has a size on the order of micrometers (e.g., 10-400 micrometers). In this way, biochips 102 may be densely arranged to provide sensing of different analytes in a small physical space. The biochips 102 may also be distributed in any pattern such as, but not limited to, a rectangular array, a checkerboard pattern, or a hexagonal array. The distance between biochips 102 (or sensing regions 106 thereof) may be fixed or may vary. In any case, since each specific structured biochip 102 is functionalized by spotting or other method then detecting the whole array or portion of the array can test for multiple targets simultaneously or in sequence.

However, it is contemplated herein that functionalizing different biochips 102 with different binding molecules may be challenging when the sets are physically close on a substrate 126. In some embodiments, multiple biochips 102 suitable for sensing using any of the techniques as disclosed herein are fabricated, diced, and separately functionalized. For example, many biochips 102 of the same or different design may be fabricated on one or more substrates 126 (e.g., as dies) and then diced using known techniques. Further, since the size of each structured biochip 102 may be on the order of micrometers (e.g., 10-400 micrometers), extra space may be provided surrounding the structured biochip 102 to increase the overall size as necessary for a selected application or dicing technique.

After dicing, the biochips 102 (or groups thereof) may be functionalized using bulk processes (e.g., in vials), which can provide substantial savings in cost and time. After functionalization, the biochips are arranged on a common substrate in any suitable pattern for analysis using any of the techniques disclosed herein. In some cases, the biochips 102 are arranged with sufficient space to avoid interference or crosstalk between the corresponding reflected light.

In this way, the interferogram of the biochips 102 may be considered to be codes and the functionalization (e.g., in the vial) makes the biosensor unique for a specific target. The structured biochips are thus “encoded”. The “interferogram code” of each or all biochips 102 may be measured simultaneously before and after exposure (or potential exposure) to various analytes of interest. Further, structured biochips with different encodings and functionalized surfaces can be distinguished using any suitable technique. For example, the biochips 102 functionalized for different analytes may be distinguished based on their physical structure. As another example, the biochips 102 functionalized for different analytes may be distinguished based on reflectivity values at one or more wavelengths. As another example, software techniques may be applied to separate the information from structured biochips functionalized for different analytes. These techniques can include, but are not limited to, machine learning methods or statistical methods or learning models or Al based methods.

The herein described subject matter sometimes illustrates different components contained within, or connected with, other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “connected” or “coupled” to each other to achieve the desired functionality, and any two components capable of being so associated can also be viewed as being “couplable” to each other to achieve the desired functionality. Specific examples of couplable include but are not limited to physically interactable and/or physically interacting components and/or wirelessly interactable and/or wirelessly interacting components and/or logically interactable and/or logically interacting components.

It is believed that the present disclosure and many of its attendant advantages will be understood by the foregoing description, and it will be apparent that various changes may be made in the form, construction, and arrangement of the components without departing from the disclosed subject matter or without sacrificing all of its material advantages. The form described is merely explanatory, and it is the intention of the following claims to encompass and include such changes. Furthermore, it is to be understood that the invention is defined by the appended claims.

What is claimed:

1. A sensor comprising:
a structured biochip comprising:
a structured film disposed on a substrate, wherein the structured film includes a plurality of sensing regions spatially distributed across the substrate, wherein the plurality of sensing regions provides spatially-varying reflectivity across the substrate, wherein the structured film is functionalized with binding molecules configured to selectively bind with one or more analytes; and
a sample delivery sub-system including one or more channels configured to expose the structured film to a test substance;
an illumination sub-system including one or more illumination sources and one or more optical elements configured to direct illumination from the one or more illumination sources to the structured film;
a collection sub-system including a multi-pixel detector and one or more lenses configured to direct light from the structured film onto the multi-pixel detector in response to the illumination; and
a controller including one or more processors configured to execute program instructions causing the one or more processors to:
receive first data from the multi-pixel detector associated with optical characteristics of the structured film prior to exposure to the test substance;
receive second data from the multi-pixel detector associated with the optical characteristics of the structured film after exposure to the test substance; and
determine concentrations of the one or more analytes bound to the binding molecules based on changes of the optical characteristics in the plurality of sensing regions of the structured film using the first and second data.

2. The sensor of claim 1, wherein the optical characteristics include at least one of reflectivity or transmissivity characteristics.

3. The sensor of claim 1, wherein the plurality of sensing regions provides a plurality of thicknesses of the structured film.

4. The sensor of claim 3, wherein at least one of the plurality of sensing regions has a constant thickness.

5. The sensor of claim 3, wherein at least one of the plurality of sensing regions has a graduated thickness.

6. The sensor of claim 3, wherein the plurality of thicknesses of the structured film are in a range of 100 nanometers to 10 micrometers.

7. The sensor of claim 1, wherein the plurality of sensing regions includes two or more sets of sensing regions, wherein the one or more analytes include two or more analytes, wherein the binding molecules in each of the two or more sets of sensing regions are different and configured to bind to different analytes of the two or more analytes.

8. The sensor of claim 1, wherein each of the plurality of sensing regions includes grating structures, wherein the grating structures in different sensing regions of the plurality of sensing regions differ in at least one of a period or a duty cycle.

9. The sensor of claim 8, wherein the grating structures in at least one of the plurality of sensing regions includes elements distributed with two or more periods.

10. The sensor of claim 8, wherein the grating structures in at least one of the plurality of sensing regions includes a period smaller than a wavelength in the illumination.

11. The sensor of claim 1, wherein the collection sub-system includes an imaging sub-system configured to image the structured film onto the multi-pixel detector, wherein the first data includes one or more reference images of the structured film prior to exposure to the test substance generated with one or more spectra, wherein the second data includes one or more test images of the structured film after exposure to the test substance generated with the one or more spectra.

12. The sensor of claim 11, wherein determining the concentrations of the one or more analytes bound to the binding molecules based on reflectivity changes in the plurality of sensing regions of the structured film using first and second images comprises:
generating one or more difference images associated with a difference between the corresponding reference and test images associated with the one or more spectra; and
determining the concentrations of the one or more analytes based on the one or more difference images.

13. The sensor of claim 12, wherein the one or more spectra include one or more narrowband spectra.

14. The sensor of claim 13, wherein the one or more narrowband spectra have bandwidths less than or equal to 100 nm.

15. The sensor of claim 12, wherein determining the concentrations of the one or more analytes based on the one or more difference images comprises:
determining the concentrations of the one or more analytes based on the one or more difference images using a Fourier Analysis technique.

16. The sensor of claim 12, wherein determining the concentrations of the one or more analytes based on the one or more difference images comprises:
determining the concentrations of the one or more analytes using a machine learning model trained on a training data set including difference images generated with known concentrations of the one or more analytes on the structured film.

17. The sensor of claim 11, wherein the one or more spectra include a broadband spectrum.

18. The sensor of claim 17, wherein the broadband spectrum has a bandwidth greater than or equal to 100 nm.

19. The sensor of claim 1, wherein the illumination from the one or more illumination sources has a broadband spectrum, wherein the multi-pixel detector is a component of a spectrometer configured to capture reflection spectra of the plurality of sensing regions in response to the illumination, wherein the first data includes reference spectra of the plurality of sensing regions prior to exposure to the test substance, wherein the second data includes test spectra of the plurality of sensing regions after exposure to the test substance.

20. The sensor of claim 1, wherein the substrate comprises:
at least one of a semiconductor, a metal, or a dielectric material.

21. The sensor of claim 1, wherein the multi-pixel detector comprises:
at least one of a charge-coupled-device (CCD) or a complementary metal-oxide-semiconductor (CMOS) device.

22. The sensor of claim 1, wherein pixels of the multi-pixel detector are arranged in a two-dimensional array.

23. The sensor of claim 1, wherein pixels of the multi-pixel detector are arranged in a one-dimensional array.

24. The sensor of claim 1, wherein the sample delivery sub-system comprises a microfluidic channel.

25. A structured biochip comprising:
a structured film disposed on a substrate, wherein the structured film includes a plurality of sensing regions spatially distributed across the substrate, wherein the plurality of sensing regions provides spatially-varying optical characteristics across the substrate, wherein the structured film is functionalized with binding molecules configured to selectively bind with one or more analytes, wherein binding of the one or more analytes to the binding molecules generates an adlayer, wherein a thickness of the adlayer impacts the spatially-varying optical characteristics of the plurality of sensing regions, wherein at least one of a presence or a concentration of at least one of the one or more analytes is determinable based on the spatially-varying optical characteristics of the plurality of sensing regions.

26. The structured biochip of claim 25, wherein the plurality of sensing regions provides a plurality of thicknesses of the structured film.

27. The structured biochip of claim 25, wherein each of the plurality of sensing regions includes grating structures, wherein the grating structures in different sensing regions of the plurality of sensing regions differ in at least one of a period or a duty cycle.

28. A device comprising:
a structured biochip comprising a structured film disposed on a substrate, wherein the structured film includes a plurality of sensing regions spatially distributed across the substrate, wherein the plurality of sensing regions provides spatially-varying optical characteristics across the substrate, wherein the structured film is functionalized with binding molecules configured to selectively bind with one or more analytes, wherein binding of the one or more analytes to the binding molecules generates an adlayer, wherein a thickness of the adlayer impacts the spatially-varying optical characteristics of the plurality of sensing regions; and
a sample delivery sub-system including one or more channels configured to expose the structured film to a test substance, wherein the one or more channels are transparent to selected wavelengths of illumination, wherein the wherein at least one of a presence or a concentration of at least one of the one or more analytes is determinable based on the spatially-varying optical characteristics of the plurality of sensing regions associated with the selected wavelengths.

29. The device of claim 28, wherein the plurality of sensing regions provides a plurality of thicknesses of the structured film.

30. The device of claim 28, wherein each of the plurality of sensing regions includes grating structures, wherein the grating structures in different sensing regions of the plurality of sensing regions differ in at least one of a period or a duty cycle.
